(19) United States
(12) Patent Application Publication
(10) Pub. No.: US 2010/0041921 A1
(43) Pub. Date: Feb. 18, 2010

(54) PROCESS FOR PREPARING 3-AMINOMETHYL-3,5,5-TRIMETHYLCYCLOHEXYLAMINE

(75) Inventors: Christian Lettmann, Coesfeld (DE); Gerda Grund, Coesfeld (DE); Juergen Lippe, Gelsenkirchen (DE); Cord Knoop, Haltern (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: EVONIK DEGUSSA GmbH, Essen (DE)

(21) Appl. No.: 12/525,841

(22) PCT Filed: Jan. 25, 2008
(86) PCT No.: PCT/EP2008/050868

§ 371 Date: Aug. 5, 2009

(30) Foreign Application Priority Data
Mar. 7, 2007 (DE) 10 2007 011 483.6

(51) Int. Cl. C07C 209/04

(52) U.S. Cl. 564/445

(57) ABSTRACT

The invention relates to an improved process for preparing 3-aminomethyl-3,5,5-trimethylcyclohexylamine, referred to hereinafter as isophoronediamine or IPD for short, by aminating hydrogenation of 3-cyano-3,5,5-trimethylcyclohexanone, referred to hereinafter as isophoronenitrile or IPN for short, in the presence of a shaped Raney hydrogenation catalyst.

The invention relates to an improved process for preparing 3-aminomethyl-3,5,5-trimethylcyclohexylamine, referred to hereinafter as isophoronediamine or IPD for short, by aminating hydrogenation of 3-cyano-3,5,5-tri-methylcyclohexanone, referred to hereinafter as isophoronenitrile or IPN for short, in the presence of a shaped Raney hydrogenation catalyst.

The invention preferably comprises a first stage for at least partial reaction of IPN with ammonia to give isophoronenitrileimine and a second stage for aminating hydrogenation of the reaction mixture in the presence of a Raney fixed bed catalyst.

IPD finds use as an epoxy resin hardener, as an amine component in polyamides and as a starting component for isophorone diisocyanate, which is itself in turn a starting component for polyurethane systems. IPD is prepared industrially preferably from IPN, which can be obtained in a known manner by the addition of hydro-cyanic acid onto isophorone (for example DE 12 40 854B1 and DE 39 42 371).

Activated metal catalysts are known in chemical technology as Raney catalysts. They are used predominantly as powder catalysts in a multitude of hydrogenation reactions. Raney catalysts are prepared from an alloy of the catalytically active metal and of an alloy component soluble in alkalis. The catalytically active components used are mainly nickel, cobalt, copper and iron. For the preparation of IPD from IPN, cobalt and ruthenium catalysts are often preferred, since they have a high selectivity with regard to the formation of the desired primary diamine. The leachable alloy constituent used is predominantly aluminium, but zinc and silicon are also suitable. The so-called Raney alloy is typically finely ground, and then the leachable component is removed fully or partly by leaching with alkalis, for example sodium hydroxide solution.

Powder catalysts have the disadvantage that they can be used only in batchwise processes. Various processes which enable the preparation of activated fixed bed metal catalysts have therefore been described. Such Raney fixed bed catalysts are particularly suitable for the industrial scale preparation of IPD, since they enable a continuous process.

The patent DE 19 540 191 describes a two-stage process for preparing isophoronediamine. In this process, in the first reaction step, isophoronenitrile is converted by reaction with ammonia in the presence of an imination catalyst to the corresponding imine. In the second reaction step, hydrogenation is effected to give isophoronediamine in the presence of a shaped Raney catalyst based on cobalt, as can be obtained according to DE 43 45 265 and DE 43 35 360. The disadvantage of the process is that metallic cobalt has to be added as a binder to the catalyst. The cobalt added is of low catalytic activity compared to Raney cobalt and leads, owing to the high cost of cobalt, compared to catalysts which do not need any metallic cobalt as a binder, to high catalyst costs.

EP 880 996 describes a process for preparing IPD, in which a shaped Raney catalyst which is prepared without addition of metallic cobalt as a binder is used. To prepare these catalysts, a cobalt-aluminium alloy present as a powder is mixed with a high molecular weight polymer and optionally promoters and then shaped to shaped bodies, for example by extrusion. The shaped bodies are subsequently calcined at temperatures up to 850° C. The thermal treatment leads to the controlled decomposition of the polymer and to the formation of a fixed bed catalyst with sufficient mechanical stability. Subsequently, the activation is effected by leaching out the aluminium by means of sodium hydroxide solution. A disadvantage of this process is that a large portion of the cobalt-aluminium alloy used remains unutilized, since the leaching of the aluminium and hence activation of the catalyst proceed only in the outermost shell of the shaped body. The core of the catalyst still consists of the cobalt-aluminium alloy used and is catalytically inactive, such that a considerable portion of the relatively expensive cobalt-aluminium alloys remains unutilized and serves merely as a support for the activated Raney nickel layer.

An optimized exploitation of the Raney metal alloy used is achieved when Raney fixed bed catalysts which are present in the form of hollow bodies are used, as can be obtained according to the teaching of EP 1 068 900. To prepare the catalysts, a mixture of the desired alloy, of an organic binder and optionally of an inorganic binder is sprayed homogeneously through a moving bed of polystyrene spheres, where it coats the spheres. The coated spheres are then calcined at temperatures from 450 to 1000° C. in order to burn out the polystyrene and to sinter the metal together in order to make the hollow form more stable. After the calcination, the catalyst is activated by means of sodium hydroxide solution. The use of appropriate catalysts for the preparation of IPD is described in EP 1 216 985. The particular advantage of this type of catalysts lies in the fact that a large portion of the alloy used is catalytically active after the activation, and thus the activity of the catalyst based on the mass of alloy used is particularly high. The stock of relatively expensive alloy in the reactor can thus be minimized.

A disadvantage of the catalysts described in EP 1 216 985 is the comparatively complicated preparation process. A particularly critical phase in the preparation process is the period between the burning-out of the Styropor spheres and the formation of a stable shell. Furthermore, the catalysts, owing to their hollow body structure, have a lower fracture resistance than catalysts which have a massive core. Moreover, the catalysts have a relatively low bulk density of only 0.3 to 1.3 g/ml, which restricts their use in fixed bed reactors which are flowed through with liquid from the bottom upward, since the catalyst particles can be set in motion easily by the flowing medium, which enhances the deactivation through mechanical attrition.

It is an object of the present invention to develop a process for preparing isophoronediamine from isophoronenitrile, in which Raney hydrogenation catalysts which contain a minimum amount of metal alloy are used, and equal or better IPD yields are nevertheless achieved compared to processes known to date in which Raney hydrogenation catalysts are used.

It has now been found that, surprisingly, the problem of interest can be solved by using the catalysts described in the document PCT/EP/2005/009656. This observation is surprising in that it cannot necessarily be assumed that the required IPD yields are achieved when the catalysts described in PCT/EP/2005/009656 are used in the specific case of the aminating hydrogenation of IPN to IPD.

PCT/EP/2005/009656 describes Raney fixed bed catalysts which can be obtained by applying, especially spraying, a Raney alloy onto a support, for example silicon dioxide or aluminium oxide. The application, especially spraying, of the alloy onto the support affords shaped bodies in which only the outer shell consists of the alloy, while the inner core of the shaped bodies consists of the support material used. The use of the support material minimizes the specific use of relatively expensive alloy. The shaped bodies are activated in a known manner by treatment with acid or alkali. The advantages of the catalysts described in PCT/EP/2005/009656 over the hollow bodies of EP 1 068 900 are the less complicated production and hence reduced production costs, a higher mechanical stability and a greater variability of the bulk density.

The invention provides a process for preparing isophoronediamine by aminating hydrogenation of isophoronenitrile or isophoronenitrileimine or mixtures in the presence of at least ammonia and hydrogen, in which a shaped Raney-type hydrogenation catalyst which has been prepared by a production process comprising the following steps is used:
1) preparing the catalyst precursor by applying a pulverulent alloy to a support material, the alloy consisting at least of one active metal, and a second leachable alloy component selected from aluminium, silicon and zinc,
2) optionally drying and calcining the shaped bodies obtained in step 1),
3) activating the shaped bodies obtained in step 1) or 2) by means of acid and/or alkali,
4) optionally further modifying the catalyst activated in step 3), for example by applying metals and/or metal salts and/or acids or bases, and/or by treatment in reducing or oxidizing atmosphere.

The catalysts to be used in accordance with the invention are obtainable by the process described in PCT/EP/2005/009656.

The catalyst precursor is prepared by applying one alloy powder or else a plurality of alloy powders to a support material. The supports may be a wide variety of different materials, for example inorganic oxides, for example alumina, silica, silica-alumina, magnesia, zinc oxide, titanium dioxide, zirconium dioxide and mixtures of these oxides. Other inorganic materials such as ceramics, shaped bodies composed of metals, glass spheres, activated carbon, silicon carbide, calcium carbonate and barium sulphate are also suitable.

In a preferred embodiment of the invention, alloys based on cobalt/aluminium and/or nickel/aluminium, more preferably based on cobalt/nickel/aluminium, and supports based on alumina, silica and alumina-silica are used. It is advantageous when the support has a very low pore volume and a relatively inert surface in order to prevent side reactions proceeding on the support material.

The alloy is applied to the support preferably by spray application of a liquid suspension at least comprising the alloy powder(s) and optionally one or more of the following components: inorganic binders (e.g. Ni, Co, Fe, Cu, other metal powders or inorganic powders), organic binders (e.g. polyvinyl alcohol), water, promoters, pore formers. The particle size of the pulverulent alloy is in the range from 1 to 200 μm. The suspension can be applied to the support, for example, in a drum or a spray chamber, in which case elevated temperature can be employed, such that liquid introduced, for example water, is removed actually during this preparation step.

In some cases, pretreatment of the support is necessary in order to improve the adhesion of the alloy to be applied. Suitable processes are those in which the surface of the support is roughened or modified, for example by an acid treatment or by etching processes. In some cases, it may be advantageous to initially cover the support, to modify its surface properties, with a material which acts as a kind of binder between support material and alloy. The binders used may, for example, be inorganic oxides such as aluminium oxide, titanium dioxide or metal powder.

Optionally, the resulting catalyst precursors are dried further and calcined in an additional process step, preferably at temperatures from 100 to 1200° C., more preferably from 100 to 1000° C.

The catalysts used in accordance with the invention may also consist of several layers. The catalyst precursors are preferably then dried between the individual coating steps, preferably at temperatures from 60 to 150° C.

The catalyst precursor is activated by leaching out the soluble alloy components, preferably with an aqueous mineral base, for example sodium hydroxide solution. Subsequently, the activated catalyst is washed with water.

The mass ratio of leachable alloy component to active metal component in the alloy is preferably in the range of 20:80 to 80:20. The catalysts to be used in accordance with the invention comprise, in addition to the leachable alloy component and the active metal component, preferably further doping elements or promoters selected from the group of transition metal groups IIIb to VIIb and VIII, including the rare earths. Main group elements and compounds thereof, especially those of main groups 1 and 2, are also suitable as promoters. The doping of Raney-type catalysts is described, for example, in documents U.S. Pat. No. 4,153,578, DE 21 01 856, DE 21 00 373 or DE 20 53 799. Preferred promoters are magnesium, chromium, manganese, iron, cobalt, vanadium, tantalum, titanium, cerium, tungsten, rhenium, platinum, palladium, ruthenium, nickel, copper, silver, gold and/or molybdenum. Very particular preference is given to magnesium, chromium and/or nickel. The promoters can be added as an alloy constituent and/or at any time during the preparation, for example not until after the activation step. The promoters may be added either in elemental form or in the form of their compounds. The proportion of promoters in the catalyst is up to 20% based on the total weight of the catalyst, preferably between 2 and 10%.

The bulk density of the catalysts can be set within a wide range from 0.8 to 3 g/ml and is dependent especially on the bulk density of the support material and on its proportion by mass in the catalyst, i.e. the ratio of support mass to the total mass of the catalyst.

In the process according to the invention for preparing isophoronediamine, the catalysts described are used for the step of aminating hydrogenation of isophoronenitrile or isophoronenitrileimine. This process can be carried out batchwise or continuously.

It is possible to carry out the process according to the invention in one stage or in several stages. When the process is carried out in one stage, isophoronenitrile is hydrogenated under aminating conditions directly in the presence of ammonia, hydrogen, the shaped hydrogenation catalyst and if desired further additives, and in the presence or absence of organic solvents. The term “in several stages” means that, first, in a separate reactor or reactor section, isophoronenitrile is first converted completely or partly to isophoronenitrileimine, and this isophoronenitrileimine is hydrogenated under aminating conditions in the presence of at least ammonia as a pure substance or in a mixture with other components.

A preferred embodiment of the process according to the invention for preparing IPD is a two-stage process: in the first stage, at least a portion of the IPN used, in the presence or absence of an imination catalyst and/or of solvents, is converted to isophoronenitrileimine by reaction with ammonia. The ratio of isophoronenitrileimine to isophoronenitrile should, after the imination, be greater than 1, preferably greater than 4 and most preferably greater than 9. In the second stage, the reaction product of the first stage, as obtained or after a further treatment and/or addition of further ammonia, is hydrogenated under aminating conditions over the catalysts to be used in accordance with the invention in the presence of at least ammonia and hydrogen and in the presence or absence of an organic solvent, at a temperature of 20 to 150° C., preferably 40 to 130° C., and a pressure of 0.3 to 50 MPa, preferably 5 to 30 MPa.

In a further preferred embodiment, the conversion of IPN to IPD is effected in three separate reaction chambers. In the first reaction chamber, IPN is converted to isophoronenitrileimine with excess ammonia over imine formation catalysts at temperatures from 20 to 150° C. and pressures of 5 to 30 MPa. In the second reaction chamber, the reaction products formed are hydrogenated with hydrogen in the presence of excess ammonia over the catalysts to be used in accordance with the invention at temperatures from 20 to 130° C. and pressures of 5 to 30 MPa. In the third reaction chamber, the reaction products formed are hydrogenated over the catalysts to be used in accordance with the invention at temperatures from 100 to 160° C. and pressures of 5 to 30 MPa.

In order to accelerate the establishment of equilibrium in the imination reaction, it is appropriate to use an imination catalyst. For this purpose, the imination catalysts known from the prior art may be used. Suitable catalysts are, for example, inorganic or organic ion exchangers (see EP 042 119), supported heteropolyacids (see DE 44 26 472), acidic metal oxides, especially aluminium oxide and titanium dioxide (see EP0449089), organopolysiloxanes containing sulphonic acid groups (DE 19 627 265.3) and acidic zeolites. When an imination catalyst is used, the reaction temperature may be from 10 to 150° C., preferably from 30 to 130° C. and most preferably from 40 to 100° C. The pressure is from the autogenous pressure of the mixture to 50 MPa. The imination reaction is preferably carried out at the pressure at which the subsequent reductive amination is also carried out.

Even though the imination of isophoronenitrile with liquid ammonia is preferably carried out without the addition of further solvents, it is also possible to work in the presence of additional solvents. Suitable solvents include monohydric alcohols having 1 to 4 carbon atoms, especially methanol, and ethers, particularly THF, MTBE and dioxane.

In the imination stage, from 1 to 500 mol, preferably from 5 to 200 mol, more preferably from 5 to 100 mol, of ammonia are used per mole of IPN used. Typical catalyst hourly space velocities are in the range of 0.01 to 10 kg of IPN per kg of catalyst and hour, preferably 0.5 to 10 kg and more preferably 0.5 to 5 kg of IPN per kg of catalyst and hour.

In the case of imination in the presence of an imination catalyst, the catalyst may be present in the form of a suspension catalyst or fixed bed catalyst. It is advantageous to use fixed bed catalysts. In a particularly preferred embodiment, IPN and ammonia are passed through a reaction tube filled with imination catalyst continuously from the bottom upward.

The imination catalyst is preferably arranged in a dedicated reactor. However, it is also possible to arrange the imination catalyst together with the catalyst used for the aminating hydrogenation in the same reactor.

Apart from the aforementioned constituents of the mixture to be fed to the imination stage, the mixture may additionally comprise fractions having higher or lower boiling points than IPD from the distillative workup of the reaction mixture drawn off from the trickle bed reactor. Such fractions may, apart from residues of IPD, also contain those by-products from which IPD is formed again under reaction conditions. Recycling of such fractions allows the yield of IPD to be increased significantly. It is particularly advantageous to recycle the fraction which boils above IPD and, apart from residues of IPD, also contains 3,3,5-trimethyl-6-imino-7-azabicyclo[3.2.1]octane as the main product. It is likewise particularly advantageous to recycle fractions comprising incompletely converted IPN, especially isophoroneaminonitrile. The recyclings can also, if desired, be added directly to the reaction mixture to be fed to the hydrogenation stage.

The crucial improvement in the process according to the invention consists in the use of the shaped Raney hydrogenation catalyst already described in the aminating hydrogenation. In the preferred two-stage process, a mixture comprising isophoronenitrileimine is hydrogenated with the aid of the shaped hydrogenation catalyst. The mixture fed to the hydrogenation stage may immediately be any which is obtained in the imination of IPN with ammonia in the first stage, or as obtained after addition or removal of components, for example ammonia, organic solvents, bases, cocatalysts and/or water. Preference is given to performing the hydrogenation continuously in fixed bed reactors which can be operated in trickle mode or liquid-phase mode. Suitable reactor types are, for example, shaft ovens, staged reactors or tube bundle reactors. It is also possible to connect a plurality of fixed bed reactors in series for the hydrogenation, in which case each of the reactors is operated in trickle bed mode and liquid-phase mode as desired.

It is preferred that the hydrogenation catalysts to be used in accordance with the invention are first conditioned with ammonia before they are used in the hydrogenation. To this end, the catalysts are contacted with ammonia or with mixtures of ammonia and one or more solvents. Preference is given to effecting the conditioning after the catalysts have been installed in the hydrogenation reactor, but it can also be effected before installation of the catalysts. For conditioning, from 0.2 to 3 m³, preferably from 0.5 to 2 m³ of ammonia are used per m³ of catalyst and hour. Typically, temperatures from 20 to 150° C., preferably 40 to 130° C., are employed. Particular preference is given to passing through a temperature ramp in which the catalyst, beginning at moderately elevated temperature, preferably from 20 to 50° C., is heated slowly to the reaction temperature desired later for the hydrogenation, preferably 20 to 150° C. The conditioning is preferably carried out in the presence of hydrogen, in which case the partial pressure of the hydrogen used in the reactor encompasses the range of 0.1 to 30 MPa, preferably 5 to 25 MPa, more preferably 10 to 20 MPa.

The duration of the conditioning depends on the amount of ammonia used and is preferably from 1 to 48 h, more preferably from 12 to 24 h.

The hydrogen required for the hydrogenation can be added to the reactor either in excess, for example at up to 10 000 molar equivalents, or only in such an amount that the hydrogen consumed by reaction and the portion of the hydrogen which leaves the reactor dissolved in the product stream are replenished. In continuous mode, the hydrogen can be fed in in cocurrent or countercurrent.

In a preferred embodiment, the hydrogenation over the catalysts to be used in accordance with the invention is effected in liquid ammonia as the solvent. From 1 to 500 mol, preferably from 5 to 200 mol, more preferably from 5 to 100 mol of ammonia are used per mole of IPN. Appropriately, at least the amount of ammonia which has been established in the preceding imination is used. However, the ammonia content can also be increased to the desired value before the hydrogenation by adding additional ammonia.

The hydrogenation is effected typically at temperatures from 20 to 150° C., preferably from 40 to 130° C., and pressures of 0.3 to 50 MPa, preferably 5 to 30 MPa.

It is also possible to carry out the hydrogenation in the presence of the solvents already mentioned for the imination stage. The significant advantage of using a solvent is that the hydrogenation can be carried out at lower pressures from 0.3 to 10 MPa.

In the hydrogenation of IPN or isophoronenitrileimine, two different stereoisomers can be formed. The selection of a temperature profile in the hydrogenation step can influence the isomer ratio. It is, for example, possible to partly hydrogenate a mixture comprising IPN or isophoronenitrileimine first at a temperature from 20 to 90° C., and then to complete the reaction in a second step within a temperature range from 90 to 150° C. By virtue of the maintenance of relatively low reaction temperatures in the 1st step, the selectivity can be shifted in favour of the cis isomer. The maintenance of relatively low reaction temperatures at the start of the reaction additionally has the advantage that the thermally labile isophoronenitrileimine is hydrogenated particularly gently and, for example, the elimination of hydrocyanic acid is minimized. The isophoroneaminonitrile formed as an intermediate is significantly more thermally stable and can therefore be hydrogenated at higher temperatures without any risk of elimination of hydrocyanic acid.

The desired temperature profile can be implemented, for example, through the series connection of two or more separately heatable reactors. However, it is also possible to implement a rising temperature profile in only one hydrogenation reactor. Particular preference is given to carrying out the hydrogenation reaction in an adiabatic trickle bed reactor, in which the reaction mixture is fed to the reactor at temperatures from 20 to 90° C., and leaves it again at from 90 to 150° C. owing to the heat of reaction which occurs and is absorbed by the reaction mixture.

The required volume of the catalysts to be used in accordance with the invention is guided by the LHSV (liquid hourly space velocity) which depends on the operating pressure, the temperature, the concentration and the catalyst activity and has to be maintained in order to ensure substantially complete hydrogenation of the IPN used. Typically, the LHSV, when the mixture of IPN, ammonia and hydrogen to be used with preference is used, is from 0.5 to 4 m³ of IPN/ammonia mixture per m3 of catalyst and hour, preferably from 1 to 3 m³ (m³×h).

The reaction mixture leaving the hydrogenation reactor is worked up in a manner known per se. This workup typically comprises removal of the ammonia, of the solvents or mixtures of solvent and ammonia, if solvent is present, and isolation of the IPD.

Irrespective of whether the process according to the invention is performed in a preferred embodiment or not, one or more hydroxide bases can also be added in the case of reaction of a mixture of IPN, ammonia, hydrogen and optionally solvent. The addition of hydroxide bases can increase the yield of IPD by reducing by-product formation. The hydroxide base is preferably added before the hydrogenation step, but it can also be added actually before the imination. Suitable hydroxide bases are, for example, alkali metal hydroxides or alkaline earth metal hydroxides. Particularly preferred hydroxide bases are quaternary ammonium hydroxides of the general formula (R¹R²R³R⁴N)OH where R¹ to R⁴ may be the same or different and are each aliphatic, cycloaliphatic or aromatic radicals. Examples are tetramethyl-, tetraethyl-, tetra-n-propyl- and tetra-n-butylammonium hydroxide. Suitable concentrations are 0.01 to 100 mmol, preferably 0.05 to 20 mmol, of a tetraalkylammonium hydroxide per mole of IPN.

It is also possible to use one or more cocatalysts in the process according to the invention. Suitable cocatalysts are salts of cobalt, nickel, lanthanum, cerium or yttrium, preferably salts of cobalt and nickel.

### EXAMPLES

### Inventive Catalyst

A coating solution is prepared by suspending 776 g of a Co/Al/Cr/Ni alloy in 700 g of water which contains magnesium nitrate and polyvinyl alcohol.

This suspension is then sprayed onto 1350 ml of glass spheres with a mean diameter of 1.5 to 2 mm. To this end, the glass spheres are first suspended in an air stream directed upward and preheated to approx. 80° C. Subsequently, the suspension is sprayed on, a temperature of approx. 90° C. being established in the course of the spraying operation in order to evaporate the water introduced.

After coating of the glass spheres with the aforementioned solution, the spheres are dried further at a temperature of approx. 90° C. in air flowing upward. In a second step, 1350 ml of the dried coated glass spheres are then coated with a further alloy solution.

A coating solution is prepared by suspending 675 g of a Co/Al/Cr/Ni alloy in 607 g of water having a content of 2% by weight of polyvinyl alcohol.

The suspension is then sprayed onto the already precoated glass spheres under the same conditions as described above.

After the second coating step, the coated glass spheres are heated to 900° C. in a nitrogen/air stream in order to burn out the polyvinyl alcohol and to sinter the alloy particles together. The spheres are then activated in a 20% by weight sodium hydroxide solution at 90° C. for 1.5 h.

The activated spheres had a diameter of approx. 3.5 mm and a coating thickness of 800-900 μm.

### Comparative Catalyst

A Raney fixed bed cobalt catalyst which is present in the form of a hollow sphere was prepared according to the teachings of documents EP 1 068 900 and EP 1 216 985.

A coating solution is prepared by suspending 800 g of a Co/Al/Cr/Ni alloy in 1000 ml of water which contains magnesium nitrate and polyvinyl alcohol.

This suspension is then sprayed onto 2000 ml of polystyrene spheres having a mean diameter of approx. 2 mm, while they are suspended in an air stream directed upward. To this end, the Styropor spheres are first suspended in an air stream directed upward and heated to approx. 80° C. Subsequently the suspension is sprayed on, a temperature of approx. 90° C. being established in the course of the spraying operation in order to evaporate the water introduced.

After the coating of the polystyrene spheres with the aforementioned solution, the spheres are dried in air flowing upward at temperatures up to 90° C.

In a second step, 1000 ml of these dried coated polystyrene spheres are then coated further with an alloy solution. The solution for the second layer consists of 800 g of a Co/Al/Cr/Ni alloy which is suspended in 1000 ml of an aqueous solution of magnesium nitrate and polyvinyl alcohol.

The suspension is then sprayed onto the already precoated Styropor spheres under the same conditions as described above.

After the second coating step, the coated polystyrene spheres are heated in a nitrogen/air stream to 900° C. in order to burn out the polystyrene and to sinter the alloy particles together. The hollow spheres are then activated in a 20% by weight sodium hydroxide solution at 80° C. for 1.5 h. The resulting activated hollow spheres have diameters in the region of 3 mm and a coating thickness of approx. 700 μm.

Aminating Hydrogenation of IPN with Inventive Catalyst and Comparative Catalyst

The test apparatus consisted of a fixed bed reactor filled with 50 ml of ion exchanger according to EP 042 119 to catalyse the imine formation from IPN and ammonia, and a downstream fixed bed reactor filled with 50 ml of the hydrogenation catalyst. For better homogenization of the liquid distribution in the hydrogenation reactor, the catalyst bed was diluted with silicon carbide of particle size <300 μm. To condition the catalyst, 100 ml/h (60 g/h) of ammonia were passed over the fixed bed at 100° C. During the conditioning, a partial hydrogen pressure of approx. 100 bar was established. After 12 hours, the conditioning was ended. Directly after the conditioning, 135 ml/h of a solution of 14.2% by weight of IPN in ammonia were conducted in. The imination reactor was flowed through from the bottom upward (liquid-phase mode), the hydrogenation reactor from the top downward (trickle mode). A temperature of 50° C. was established in the imination reactor, and of 100° C. in the hydrogenation reactor. A regulating valve was used to keep the pressure in the hydrogenation reactor constant at 250 bar by feeding in hydrogen. The composition of the end product was determined by means of gas chromatography.

A comparison of the composition of the products when the inventive catalyst and the comparative catalyst are used is contained in Table 1.

TABLE 1

| | When the | When the |
|---|---|---|
| | inventive | comparative |
| Composition of the end | catalyst is | catalyst is |
| product (GC %)¹⁾ | used | used |
| Total isophoronediamine | 98.7 | 97.6 |
| Total isophoroneaminonitrile | 0.18 | 0.09 |
| Imine | 0.26 | 0.6 |
| (2-aza-4,6,6-trimethyl- | | |
| bicyclo[3.2.1]octane) | | |
| Amidine | 0.34 | 0.75 |
| (3,3,5-trimethyl-6-amino- | | |
| 7-azabicyclo[3.2.1]octane) | | |
| Others | 0.52 | 0.96 |

¹⁾In the two end products, approx. 9.7% water which is not detected by the GC is additionally present.

The tests show that the IPD yield with the catalysts to be used in accordance with the invention is approx. 1% higher than when the comparative catalyst is used.

1. A process for preparing isophoronediamine, comprising aminating hydrogenation of isophoronenitrile, isophoronenitrileimine, or a mixture thereof in the presence of at least ammonia and hydrogen, wherein said aminating is carried out in the presence of a shaped Raney-type hydrogenation catalyst which is prepared by:
1) preparing a catalyst precursor by applying a pulverulent alloy to a support material, wherein the alloy comprises one active metal, and a second leachable alloy component selected from the group consisting of aluminum, silicon and zinc to form the shaped catalyst,
2) optionally drying and calcining the shaped catalyst obtained in 1), and
3) activating the shaped catalyst obtained in 1) or 2) by at least one of acid and alkali.

2. The process according to claim 1,
the active metal is at least one of group VIII and Ib of the Periodic Table.

3. The process according to claim 1, wherein
the active metal is at least one of cobalt, nickel, iron and copper.

4. The process according to claim 1, wherein
the alloy comprises at least one of cobalt/aluminium and nickel/aluminium.

5. The process according to claim 1, wherein
the pulverulent alloy comprises at least one of an inorganic or organic binder, promoter, acid and base.

6. The process according to claim 1, wherein
the pulverulent alloy comprises a dopant metal.

7. The process claim 5, wherein
at least one of the promoter and dopant metal is a compound selected from the group consisting of the Periodic Table: IIa, IIIb, IVb, Vb, VIb, VIIb, VIII, Ib, IIb, IIIa, IVa and Va.

8. The process according to claim 5, wherein
the promoter is selected from the group consisting of magnesium, chromium, manganese, iron, cobalt, vanadium, tantalum, titanium, cerium, tungsten, rhenium, platinum, palladium, ruthenium, nickel, copper, silver, gold and molybdenum.

9. The process according to claim 1, wherein
the support material is at least one of alumina, silica, silica-alumina, magnesia, zinc oxide, titanium dioxide, zirconium dioxide, a mixture thereof, a ceramic, a shaped body of a metal, a glass sphere, activated carbon, silicon carbide, calcium carbonate and barium sulphate.

10. The process according to claim 1, wherein
the support material comprises aluminium, silica and alumina-silica.

11. The process according to claim 5, wherein
the inorganic binder is at least one of a metal powder and an organic powder.

12. The process according to claim 5, wherein
the organic binder is polyvinyl alcohol.

13. The process according to claim 1, wherein
a particle size of the pulverulent alloy is in the range from 1 to 200 μm.

14. The process according to claim 1, wherein
the catalyst activated in 3) is further modified.

15. The process according to claim 1, wherein
the pulverulent alloy is applied to a support material by spray application.

16. The process according to claim 1, wherein
the pulverulent alloy is applied by spraying from a liquid suspension.

17. The process according to claim 1, wherein
the catalyst is conditioned before the hydrogenation with ammonia.

18. The process according to claim 1, wherein
the process is a batchwise or continuous, single-stage or multistage process.

19. The process according to claim 1, wherein
4) the catalyst activated in 3) is modified further, by applying at least one of metals, metal salts, acids, and bases, by treatment in reducing or oxidizing atmosphere, or by a combination thereof.

20. The process according to claim 1, wherein
in a first stage, at least a portion of the isophoronenitrite used is converted to isophoronenitrileimine in the presence or absence of ammonia,
in a second stage, the reaction product of the first stage, as obtained or after a further treatment and optionally further addition of ammonia, in the presence of at least ammonia and hydrogen and in the presence or absence of an organic solvent, is hydrogenated under aminating conditions over the shaped catalyst at a temperature of 20 to 150° C., and a pressure of 0.3 to 50 MPa.

21. The process according to claim 20,
wherein the conversion of isophoronentrite to isophoronediamine is effected in three separate reaction chambers:
in the first reaction chamber, isophoronentrite is converted to isophoronenitrileimine with excess ammonia over an imine formation catalyst at a temperature from 20 to 150° C. and a pressure from 5 to 30 MPa;
in the second reaction chamber, the reaction products formed are hydrogenated with hydrogen in the presence of excess ammonia over the shaped catalyst at a temperature from 20 to 130° C. and a pressure of 5 to 30 MPa;
in the third reaction chamber, the reaction products formed are hydrogenated over the shaped catalyst at a temperature from 100 to 160° C. and a pressure of 5 to 30 MPa.
